(19) United States
(12) Patent Application Publication
(10) Pub. No.: US 2021/0395249 A1
(43) Pub. Date: Dec. 23, 2021

(54) NEW ALKOXYAMINOPYRIDINE DERIVATIVES FOR TREATING PAIN AND PAIN RELATED CONDITIONS

(71) Applicant: ESTEVE PHARMACEUTICALS, S.A., Barcelona (ES)

(72) Inventors: Carmen ALMANSA-ROSALES, Barcelona (ES); Félix CUEVAG-CORDOBÉS, Madrid (ES)

(21) Appl. No.: 17/288,934

(22) PCT Filed: Nov. 4, 2019
(86) PCT No.: PCT/EP2019/080058

§ 371 Date: Apr. 27, 2021

(30) Foreign Application Priority Data
Nov. 2, 2018 (EP) 18382780.7

(51) Int. Cl. C07D 471/04

(52) U.S. Cl. CPC C07D 471/04

(57) ABSTRACT

The present invention relates to new compounds of formula (I) that show dual activity towards the subunit α2δ of voltage-gated calcium channels (VGCC), especially the α2δ-1 subunit of voltage-gated calcium channels, and the noradrenaline transporter (NET). The invention is also related to the process for the preparation of said compounds as well as to compositions comprising them, and to their use as medicaments.

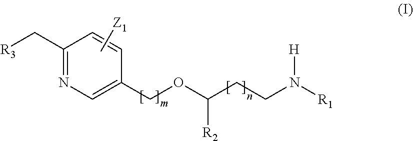

### FIELD OF THE INVENTION

The present invention relates to new compounds that show dual activity towards the subunit α2δ of voltage-gated calcium channels (VGCC), especially the α2δ-1 subunit of voltage-gated calcium channels, and the noradrenaline transporter (NET). The invention is also related to the process for the preparation of said compounds as well as to compositions comprising them, and to their use as medicaments.

### BACKGROUND OF THE INVENTION

The adequate management of pain represents an important challenge, since currently available treatments provide in many cases only modest improvements, leaving many patients unrelieved (Turk, D. C. Wilson, H. D., Cahana, A.; 2011; Lancet 377; 2226-2235). Pain affects a big portion of the population with an estimated prevalence of 20% and its incidence, particularly in the case of chronic pain, is increasing due to the population ageing. Additionally, pain is clearly correlated to comorbidities, such as depression, anxiety and insomnia, which leads to important productivity losses and socio-economical burden (Goldberg, D. S., McGee. S. J., 2011; BMC Public Health; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al.; Pharmacol. Rev.; 2015; 67; 821-870). The VGCC are assembled through interactions of different subunits, namely α1 (Caᵥα1), β (Caᵥβ) α2δ (Caᵥα2δ) and γ (Caᵥγ). The α1 subunits are the key porous forming units of the channel complex, being responsible for Ca²⁺ conduction and generation of Ca²⁺ influx. The α2δ, β, and γ subunits are auxiliary, although they are very important for the regulation of the channel since they increase the expression of the α1 subunits in the plasma membrane as well as modulate their function resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N-(Caᵥ2.2), P/Q-(Caᵥ2.1), and R-(Caᵥ2.3) types, depending on the channel forming Caᵥα subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo; Neurotherapeutics; 2009; 6; 679-692; Zamponi et al., 2015; Neumaier et al.; Prog. Neurobiol.; 2015; 129; 1-36). A large body of data has clearly indicated that VGCC are implicated in mediating various disease states including pain processing. Drugs interacting with the different calcium channel subtypes and subunits have been developed. Current therapeutic agents include drugs targeting the L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of the N-type (Caᵥ2.2) calcium channels, has been approved as a treatment of intractable pain.

The Caᵥ1 and Caᵥ2 subfamilies contain an auxiliary subunit which is the therapeutic target of the gabapentinoid drugs of value in certain epilepsies and chronic neuropathic pain (Perret and Luo, 2009; Vink and Alewood; British J. Pharmacol.; 2012; 167; 970-989). To date, there are four known α2δ subunits, each encoded by a unique gene and all possessing splice variants. Each α2δ protein is encoded by a single messenger RNA and is post-translationally cleaved and then linked by disulfide bonds. Four genes encoding the α2δ subunits have now been cloned. The α2δ-1 was initially cloned from skeletal muscle and shows a fairly ubiquitous distribution. The α2δ-2 and α2δ-3 subunits were subsequently cloned from brain. The most recently identified subunit, the α2δ-4, is largely non-neuronal. The human α2δ-4 protein sequence shares 30, 32 and 61% identity with the human α2δ-1, α2δ-2 and α2δ-3 subunits, respectively. The gene structure of all the α2δ subunits is similar. All the α2δ subunits show several splice variants (Davies et al.; Trends Pharmacol. Sci.; 2007; 28; 220-228; Dolphin, A. C.; Nat. Rev. Neurosci.; 2012; 13; 542-555; Dolphin, A. C.; Biochim. Biophys. Acta; 2013; 1828; 1541-1549).

The Caᵥα2δ-1 subunit may play an important role in neuropathic pain development (Perret and Luo, 2009; Vink and Alewood, 2012). Biochemical data have indicated a significant Caᵥα2δ-1, but not a Caᵥα2δ-2, subunit upregulation in the spinal dorsal horn, and DRG (dorsal root ganglia) after nerve injury that correlates with neuropathic pain development. In addition, blocking axonal transport of injury-induced DRG Caᵥα₂δ-1 subunit to the central presynaptic terminals diminishes tactile allodynia in nerve injured animals, suggesting that elevated DRG Caᵥα2δ-1 subunit contributes to neuropathic allodynia.

The Caᵥα2δ-1 subunit (and the Caᵥα2δ-2, but not the Caᵥα2δ-3 and the Caᵥα2δ-4, subunits) is the binding site for gabapentin which has anti-allodynic/hyperalgesic properties in patients and animal models. Because the injury-induced Caᵥα2δ-1 expression correlates with neuropathic pain, development and maintenance, and various calcium channels are known to contribute to spinal synaptic neurotransmission and DRG neuron excitability, the injury-induced Caᵥα2δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα2δ-1 subunit can block nerve injury-induced Caᵥα2δ-1 upregulation and prevent the onset of allodynia and reserve established allodynia.

As above mentioned, the α2δ subunits of VGCC form the binding site for gabapentin and pregabalin which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα2δ-1 subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009; Vink and Alewood, 2012, Zamponi et al., 2015).

It is also known that Noradrenaline (NA), also called norepinephrine, functions in the human brain and body as a hormone and neurotransmitter. Noradrenaline exerts many effects and mediates a number of functions in living organisms. The effects of noradrenaline are mediated by two distinct super-families of receptors, named alpha- and beta-adrenoceptors. They are further divided into subgroups exhibiting specific roles in modulating behavior and cognition of animals. The release of the neurotransmitter noradrenaline throughout the mammalian brain is important for modulating attention, arousal, and cognition during many behaviors (Mason, S. T.; Prog. Neurobiol.; 1981; 16; 263-303).

The noradrenaline transporter (NET, SLC6A2) is a monoamine transporter mostly expressed in the peripheral and central nervous systems. The NET recycles primarily NA, but also serotonin and dopamine, from synaptic spaces into presynaptic neurons. The NET is a target of drugs treating a variety of mood and behavioral disorders, such as depression, anxiety, and attention-deficit/hyperactivity disorder (ADHD). Many of these drugs inhibit the uptake of NA into the presynaptic cells through NET. These drugs therefore increase the availability of NA for binding to postsynaptic receptors that regulate adrenergic neurotransmission. The NET inhibitors can be specific. For example, the ADHD drug atomoxetine is a NA reuptake inhibitor (NRI) that is highly selective for NET. Reboxetine was the first NRI of a new antidepressant class (Kasper et al.; Expert Opin. Pharmacother.; 2000; 1; 771-782). Some NET inhibitors also bind multiple targets, increasing their efficacy as well as their potential patient population.

Endogenous, descending noradrenergic fibers impose analgesic control over spinal afferent circuitry mediating the transmission of pain signals (Ossipov et al.; J. Clin. Invest.; 2010; 120; 3779-3787). Alterations in multiple aspects of noradrenergic pain processing have been reported, especially in neuropathic pain states (Ossipov et a., 2010; Wang et al.; J. Pain; 2013; 14; 845-853). Numerous studies have demonstrated that activation of spinal α2-adrenergic receptors exerts a strong antinociceptive effect. Spinal clonidine blocked thermal and capsaicin-induced pain in healthy human volunteers (Ossipov et al., 2010). Noradrenergic reuptake inhibitors have been used for the treatment of chronic pain for decades: most notably the tricyclic antidepressants, amitriptyline, and nortriptyline. Once released from the presynaptic neuron. NA typically has a short-lived effect, as much of it is rapidly transported back into the nerve terminal. In blocking the reuptake of NA back into the presynaptic neurons, more neurotransmitter remains for a longer period of time and is therefore available for interaction with pre- and postsynaptic α₂-adrenergic receptors (AR). Tricyclic antidepressants and other NA reuptake inhibitors enhance the antinociceptive effect of opioids by increasing the availability of spinal NA. The α₂A-AR subtype is necessary for spinal adrenergic analgesia and synergy with opioids for most agonist combinations in both animal and humans (Chabot-Doré et al.; Neuropharmacology; 2015; 99; 285-300). A selective upregulation of spinal NET in a rat model of neuropathic pain with concurrent downregulation of serotonin transporters has been shown (Fairbanks et al.; Pharmacol. Ther.; 2009; 123; 224-238). Inhibitors of NA reuptake such as nisoxetine, nortriptyline and maprotiline and dual inhibitors of the noradrenaline and serotonin reuptake such as imipramine and milnacipran produce potent anti-nociceptive effects in the formalin model of tonic pain. Neuropathic pain resulting from the chronic constriction injury of the sciatic nerve was prevented by the dual uptake inhibitor, venlafaxine. In the spinal nerve ligation model, amitriptyline, a non-selective serotonin and noradrenaline reuptake blocker, the preferential noradrenaline reuptake inhibitor, desipramine and the selective serotonin and noradrenaline reuptake inhibitors, milnacipran and duloxetine, produce a decrease in pain sensitivity whereas the selective serotonin reuptake inhibitor, fluoxetine, is ineffective (Mochizucki, D.; Psychopharmacol.; 2004; Supplm. 1; S15-S19; Hartrick, C. T.; Expert Opin. Investig. Drugs; 2012; 21; 1827-1834). A number of nonselective investigational agents focused on noradrenergic mechanisms with the potential for additive or even synergistic interaction between multiple mechanisms of action are being developed (Hartrick, 2012).

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al.; Nat. Biotechnol.; 2009; 27; 659-666).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al.; Lancet Neurol.; 2013; 12(11); 1084-1095). In fact, positive synergistic interaction for several compounds, including analgesics, has been described (Schröder et al; J. Pharmacol. Exp. Ther.; 2011; 337; 312-320; Zhang et al.; Cell Death Dis.; 2014; 5; e1138; Gilron et al., 2013).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, A. L.; Nat. Chem. Biol.; 2008, 4; 682-690).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008).

Thus, the present invention relates to dual compounds having affinity for the α2δ subunits of voltage-gated calcium channels, preferably towards the α2δ-1 subunit of voltage-gated calcium channels, which, additionally, have inhibitory effect towards the noradrenaline transporter (NET) and are, thus, more effective to treat chronic pain.

There are two potentially important interactions between the NET and the α2δ-1 subunit inhibition;
1) synergism in analgesia, thus reducing the risk of specific side effects. Preclinical research has demonstrated that gabapentinoids attenuated pain-related behaviors through supraspinal activation of the descending noradrenergic system (Tanabe et al.; J. Neuroosci. Res.; 2008; Hayashida, K.; Eur. J. Pharmacol.; 2008; 598; 21-26). In consequence, the α2δ-1-related analgesia mediated by NA-induced activation of spinal α₂-adrenergic receptors can be potentiated by the inhibition of the NET. Some evidence from combination studies in preclinical models of neuropathic pain exist. Oral duloxetine with gabapentin was additive to reduce hypersensitivity induced by nerve injury in rats (Hayashida; 2008). The combination of gabapentin and nortriptyline drugs was synergic in mice submitted to orofacial pain and to peripheral nerve injury model (Miranda, H. F. et al.; J. Orofac. Pain; 2013; 27; 301-366; Pharmacology; 2015; 95; 59-64); and
2) inhibition of pain-related affective comorbidities such as anxiety and/or depressive-like behaviors (Nicolson et al.; Harv. Rev Psychiatry; 2009; 17; 407-420). Drug modulation of the NET and the α2δ-1 subunit has been shown to produce antidepressant and anti-anxiety effects respectively (Frampton, J. E.; CNS Drugs; 2014; 28; 835-854; Hajós, M. et al.; CNS Drug Rev.; 2004; 10; 23-44).
In consequence, a dual drug that inhibited the NET and α2δ-1 subunit of VGCC may have an improved analgesic effect and may also stabilize pain-related mood impairments by acting directly on both physical pain and the possible mood alterations.

### SUMMARY OF THE INVENTION

The present invention discloses novel dual compounds with great affinity to the α2δ subunit of voltage-gated calcium channels, more specifically to the α2δ-1 subunit, and which also have inhibitory effect towards the noradrenaline transporter (NET), thus resulting in a dual activity for treating pain and pain related disorders.

The main object of the present invention is related to compounds of general formula (I):

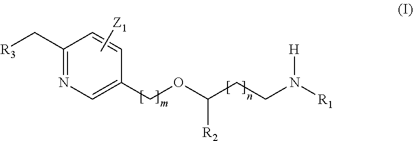

wherein

R₁ is a branched or unbranched C₁₋₆ alkyl radical or a C₁₋₆ haloalkyl radical;

R₂ is a 6-membered aryl optionally substituted by a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical or a hydroxyl radical; or 5 or 6-membered heteroaryl having at least one heteroatom selected from N, O and S;

n and m are independently 0 or 1;

Z₁ is selected from a hydrogen atom; a branched or unbranched C₁₋₆-alkyl radical; a halogen atom; a branched or unbranched C₁₋₆-alkoxy radical; a C₁₋₆-haloalkyl radical; and a C₁₋₆₋haloalcoxy radical;

R₃ represents one of the following moieties:

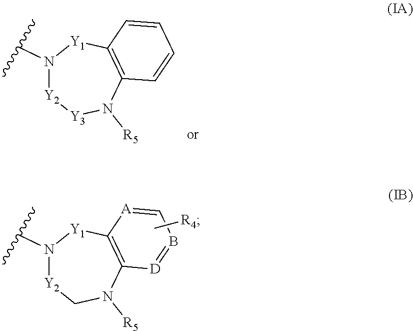

wherein

Y₁, Y₂ and Y₃ are independently —CH₂— or —C(O)—;

one or two from A, B and D represent —N— and the others are —C— or —CH—;

R₄ is a hydrogen atom, a branched or unbranched C₁₋₆-alkyl radical; a halogen atom; a branched or unbranched C₁₋₆-alkoxy radical; a C₁₋₆-haloalkyl radical; or a —NR₄ₐR_{4b} radical where R₄ₐ and R_{4b} are independently a hydrogen atom or a branched or unbranched C₁₋₆-alkyl radical;

R₅ is a hydrogen atom; a branched or unbranched C₁₋₆ alkyl radical; or a —C(O)—CH₂—NR₆ₐR_{6b} radical where R₅ₐ and R_{5b} independently represent a hydrogen atom or a branched or unbranched C₁₋₆-alkyl radical;

or a pharmaceutically acceptable salt, isomer, prodrug or solvate thereof.

It is also an object of the invention different processes for the preparation of compounds of formula (I).

Another object of the invention refers to the use of such compounds of general formula (I) for the treatment and/or prophylaxis of the α2δ-1 subunit mediated disorders and more preferably for the treatment and/or prophylaxis of disorders mediated by the α2δ-1 subunit of voltage-gated calcium channels and/or the noradrenaline transporter (NET). The compounds of the present invention are particularly suited for the treatment of pain, specially neuropathic pain, and pain related or pain derived conditions.

It is also an object of the invention pharmaceutical compositions comprising one or more compounds of general formula (I) with at least one pharmaceutically acceptable excipient. The pharmaceutical compositions in accordance with the invention can be adapted in order to be administered by any route of administration, be it orally or parenteral, such as pulmonary, nasally, rectally and/or intravenously. Therefore, the formulation in accordance with the invention may be adapted for topical or systemic application, particularly for dermal, subcutaneous, intramuscular, intra-articular, intraperitoneal, pulmonary, buccal, sublingual, nasal, percutaneous, vaginal, oral or parenteral application.

### DETAILED DESCRIPTION OF THE INVENTION

The invention first relates to compounds of general formula (I)

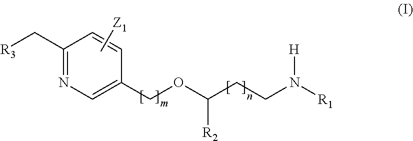

wherein:

R₁ is a branched or unbranched C₁₋₆ alkyl radical or a C₁₋₆ haloalkyl radical;

R₂ is a 6-membered aryl optionally substituted by a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical or a hydroxyl radical; or 5 or 6-membered heteroaryl having at least one heteroatom selected from N, O and S;

n and m are independently 0 or 1;

Z₁ is selected from a hydrogen atom; a branched or unbranched C₁₋₆-alkyl radical; a halogen atom; a branched or unbranched C₁₋₆-alkoxy radical; a C₁₋₆-haloalkyl radical; and a C₁₋₆₋haloalcoxy radical;

R₃ represents one of the following moieties:

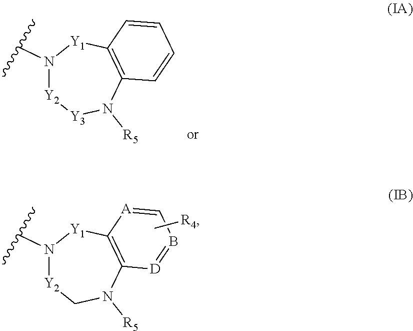

wherein
Y₁, Y₂ and Y₃ are independently —CH₂— or —C(O)—;

one or two from A, B and D represent —N— and the others are —C—or —CH—;

R₄ is a hydrogen atom, a branched or unbranched C₁₋₆-alkyl radical; a halogen atom; a branched or unbranched C₁₋₆-alkoxy radical; a C₁₋₆-haloalkyl radical; or a —NR₄ₐR_{4b} radical where R₄ₐ and R_{4b} are independently a hydrogen atom or a branched or unbranched C₁₋₆-alkyl radical;

R₅ is a hydrogen atom; a branched or unbranched C₁₋₆ alkyl radical; or a —C(O)—CH₂—NR₆ₐR_{6b} radical where R₅ₐ and R_{5b} independently represent a hydrogen atom or a branched or unbranched C₁₋₆-alkyl radical;

or a pharmaceutically acceptable salt, isomer, prodrug or solvate thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

Any compound referred to herein is intended to represent such specific compound as well as certain variations or forms. In particular, compounds referred to herein may have asymmetric centers and therefore may exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are amine-imine, amide-imidic acid, keto-enol, lactam-lactim, etc.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

A “leaving group” is a group that in a heterolytic bond cleavage keeps the electron pair of the bond. Suitable leaving groups are well known in the art and include Cl, Br, I and —O—SO₂R¹⁴, wherein R¹⁴ is F, C₁₋₄-alkyl, C₁₋₄-haloalkyl, or optionally substituted phenyl. The preferred leaving groups are Cl, Br, I, tosylate, mesylate, triflate, nonaflate and fluorosulphonate.

“Protecting group” is a group that is chemically introduced into a molecule to avoid that a certain functional group from that molecule undesirably reacts in a subsequent reaction. Protecting groups are used, among others, to obtain chemoselectivity in chemical reactions. The preferred protecting group in the context of the invention are Boc (tert-butoxycarbonyl) or Teoc (2-(trimethylsilyl)ethoxycarbonyl).

“C₁₋₆ alkyl”, as referred to in the present invention, are saturated aliphatic radicals. They may be unbranched (linear) or branched and are optionally substituted. C₁₋₆₋alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl or 1-methylpentyl. The most preferred alkyl radical are C₁₋₄ alkyl, such as methyl, ethyl, propyl, n-propyl, isopropyl, butyl, n-butyl, tert-butyl, isobutyl, sec-butyl, 1-methylpropyl, 2-methylpropyl or 1,1-dimethylethyl. Alkyl radicals, as defined in the present invention, may be optionally mono- or polysubstituted by substitutents independently selected from a halogen atom, a branched or unbranched C₁₋₆-alkoxy radical, a branched or unbranched C₁₋₆-alkyl radical, a C₁₋₆₋haloalcoxy radical, a C₁₋₆-haloalkyl radical, a trihaloalkyl radical, a hydroxyl radical and an amino radical such as —NR₄ₐR_{4b} radical.

“C₁₋₆ alkoxy” as referred to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy.

“C₃₋₆ Cycloalkyl” as referred to in the present invention, is understood as meaning saturated and unsaturated (but not aromatic), cyclic hydrocarbons having from 3 to 6 carbon atoms which can optionally be unsubstituted, mono- or polysubstituted. Examples for cycloalkyl radical preferably include but are not restricted to cyclopropyl, cyclobutyl, cyclopentyl, or cyclonexyl. Cycloalkyl radicals, as defined in the present invention, are optionally mono- or polysubstituted by substitutents independently selected from a halogen atom, a branched or unbranched radical, a branched or unbranched C₁₋₆-alkoxy radical, a C₁₋₆-haloalcoxy radical, a C₁₋₆-haloalkyl radical, a trihaloalkyl radical or a hydroxyl radical.

“Heterocycloalkyl” as referred to in the present invention, are understood as meaning saturated and unsaturated (but not aromatic), generally 5 or 6 membered cyclic hydrocarbons which can optionally be unsubstituted, mono- or polysubstituted and which have at least one heteroatom in their structure selected from N, O and S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, may be optionally mono- or polysubstituted by substitutents independently selected from a halogen atom, a branched or unbranched C₁₋₆-alkyl radical, a branched or unbranched C₁₋₆-alkoxy radical, a C₁₋₆-haloalkoxy radical, a C₁₋₆-haloalkyl radical, a trihaloalkyl radical and a hydroxyl radical. More preferably heterocycloalkyl in the context of the present invention are 5 or 6-membered ring systems optionally at least monosubstituted.

“Aryl” as referred to in the present invention, is understood as meaning ring systems with at least one aromatic ring but without heteroatoms even in only one of the rings. These aryl radicals may optionally be mono- or polysubstituted by substitutents independently selected from a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical and a hydroxyl radical, Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention is a 6-membered ring system optionally at least monosubstituted.

“Heteroaryl” as referred to in the present invention, is understood as meaning heterocyclic ring systems which have at least one aromatic ring and contain one or more heteroatoms selected from the group consisting of N, O and S and may optionally be mono- or polysubstituted by substituents independently selected from a halogen atom, a branched or unbranched C₁₋₆-alkyl radical, a branched or unbranched C₁₋₆-alkoxy radical, a C₁₋₆-haloalkoxy radical, a C₁₋₆-haloalkyl radical, a trihaloalkyl radical and a hydroxyl radical. Preferred examples of heteroaryls include but are not restricted to furan, benzofuran, pyrrole, pyridine, pyrimidine, pyridazine, pyrazine, thiophene, quinoline, isoquinoline, phthalazine, triazole, pyrazole, isoxazole, indole, benzotriazole, benzodioxolane, benzodioxane, benzimidazole, carbazole or quinazoline. More preferably heteroaryl in the context of the present invention are 5 or 6-membered ring systems optionally at least monosubstituted.

“Heterocyclic system”, as defined in the present invention, comprises any saturated, unsaturated or aromatic carbocyclic ring systems which are optionally at least mono-substituted and which contain at least one heteroatom as ring member. Preferred heteroatoms for these heterocyclyl radicals are N, S or O. Preferred substituents for heterocyclyl radicals, according to the present invention, are F, Cl, Br, I, NH₂, SH, OH, SO₂, CF₃, carboxy, amido, cyano, carbamyl, nitro, phenyl, benzyl, —SO₂NH₂, branched or unbranched C₁₋₆ alkyl and/or branched or unbranched C₁₋₆-alkoxy.

The term “ring system” according to the present invention refers to an organic system consisting of at least one ring of connected atoms but including also systems in which two or more rings of connected atoms are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions. The definition particularly includes physiologically acceptable salts, this term must be understood as equivalent to “pharmacologically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. These physiologically acceptable salts may be formed with cations or bases and, in the context of this invention, are understood to be salts formed by at least one compound used in accordance with the invention—normally an acid (deprotonated)—such as an anion, and at least one physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “solvate” is to lie understood as meaning any form of the active compound according to the invention in which this compound has attached to it via non-covalent binding another molecule (most likely a polar solvent) especially including hydrates and alcoholates, e.g. methanolate.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, or amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

In a particular and preferred embodiment R₁ is a branched or unbranched C₁₋₆ alkyl radical, more preferably methyl.

In another particular and preferred embodiment of the invention, R₂ is a phenyl radical optionally substituted by a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical or a hydroxyl radical. More preferably, the phenyl radical is unsubstituted or substituted by a halogen atom, preferably F.

In another particular and preferred embodiment of the invention, R₂ is a thienyl radical optionally substituted by a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical or a hydroxyl radical. More preferably, the thienyl radical is unsubstituted.

In another particular and preferred embodiment of the invention, R₃ is selected from:

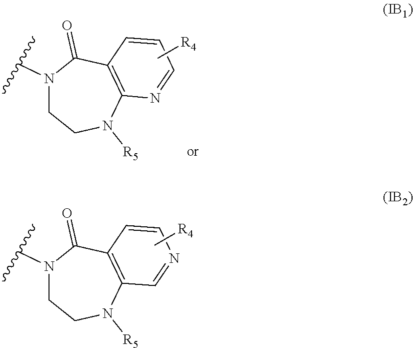

wherein R₄ and R₅ are as defined before.

In a still particular embodiment of the invention, Z₁ represents a halogen atom, more preferably fluorine or chlorine.

In another particular and preferred embodiment of the invention, R₄ is a hydrogen atom, a halogen atom, more preferable fluorine; a branched or unbranched C₁₋₆-alkoxy radical, more preferable methoxy; or a —NR₄ₐR_{4b} radical where R₄ₐ and R_{4b} are independently a hydrogen atom or a branched or unbranched C₁₋₆-alkyl radical, more preferably a hydrogen atom or methyl.

In another particular and preferred embodiment of the invention, R₅ is a branched or unbranched C₁₋₆ alkyl radical, more preferable methyl.

A particularly preferred embodiment of the invention is represented by compounds of general formula (Ia) where:

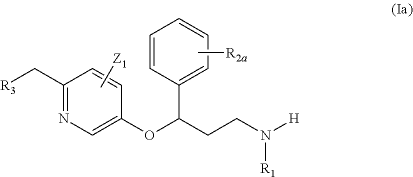

wherein R₁, R₃ and Z₁ are as defined before and R₂ₐ is selected from a hydrogen a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical and a hydroxyl radical.

A still more preferred embodiment of the invention is represented by compounds of formula (Ia):

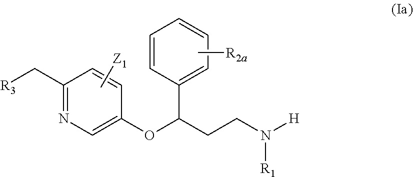

wherein

R₁ is a branched or unbranched C₁₋₆ alkyl radical, more preferably methyl;

R₂ₐ is a hydrogen atom or a halogen atom, more preferably fluorine;

R₃ is selected from:

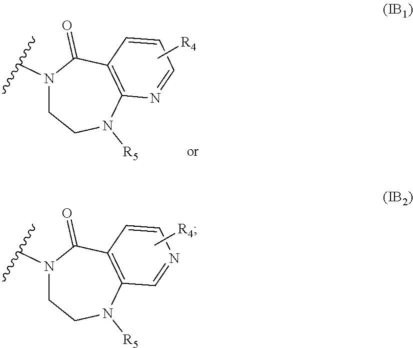

Z₁ represents a halogen atom, more preferable fluorine or chlorine;

R₄ is a hydrogen atom; a halogen atom, more preferably fluorine; a branched or unbranched C₁₋₆-alkoxy radical, more preferably methoxy; or a —NR₄ₐR_{4b} radical where R₄ₐ and R_{4b} are independently a hydrogen atom or a branched or unbranched C₁₋₆-alkyl radical, more preferably a hydrogen atom or methyl;

R₅ is a branched or unbranched C₁₋₆ alkyl radical, more preferable methyl;

or a pharmaceutically acceptable salt, isomer, prodrug or solvate thereof.

In another particularly preferred embodiment the compounds of the invention are represented by formulas (Ia1) and (Ia2):

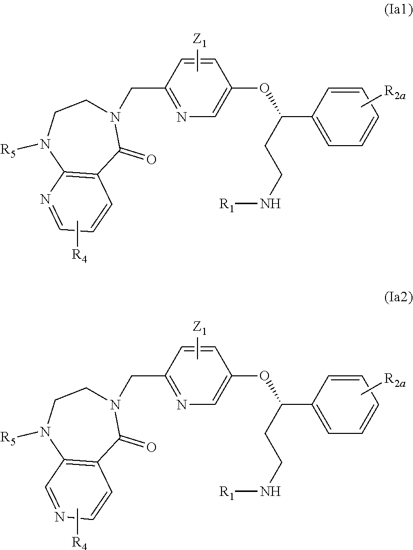

wherein R₁, R₄, R₅ and Z₁ are as defined before in this description and R₂ₐ selected from a hydrogen a halogen atom, a branched or unbranched C₁₋₆-alkyl radical, a branched or unbranched C₁₋₆-alkoxy radical a C₁₋₆₋haloalcoxy radical, a C₁₋₆-haloalkyl radical and a hydroxyl radical.

A still more preferred embodiment of the invention is represented by compounds of formula (Ia1) and (Ia2):

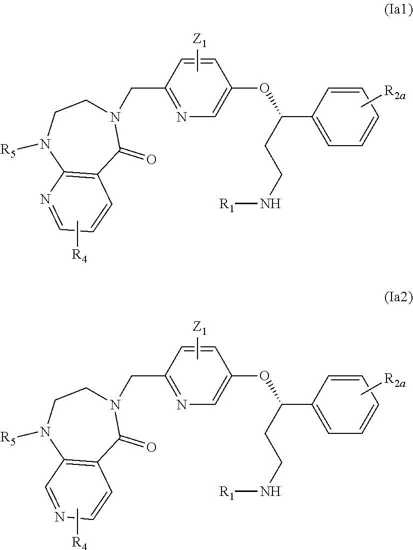

wherein

R₁ is a branched or unbranched C₁₋₆ alkyl radical, more preferably methyl;

R₂ₐ is a hydrogen atom or a halogen atom, more preferably fluorine;

Z₁ represents a halogen atom, more preferably fluorine or chlorine;

R₄ is a hydrogen atom; a halogen atom, more preferably fluorine; a branched or unbranched C₁₋₆-alkoxy radical, more preferably methoxy; or a —NR₄ₐR_{4b} radical where R₄ₐ and R_{4b} are independently a hydrogen atom or a branched or unbranched C₁₋₆-alkyl radical, more preferably a hydrogen atom or methyl;

R₅ is a branched or unbranched C₁₋₆ alkyl radical, more preferable methyl;

or a pharmaceutically acceptable salt, isomer, prodrug or solvate thereof.

In another particularly preferred embodiment the compounds of the invention are represented by formulas (Ia1′) and (Ia2′):

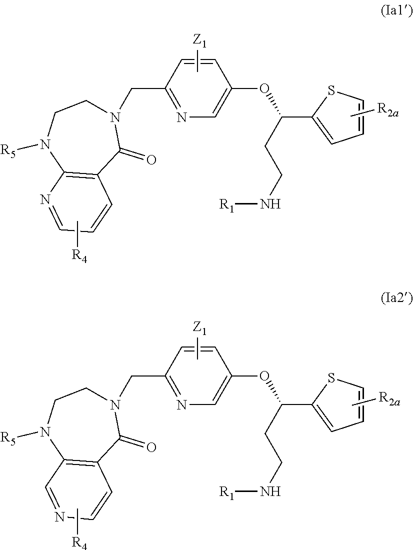

wherein R₁, R₄, R₅ and Z₁ are as defined before in this description and R₂ₐ is selected from a hydrogen a halogen atom, a branched or unbranched C₁₋₆-alkyl radical, a branched or unbranched C₁₋₆-alkoxy radical, a C₁₋₆₋haloalcoxy radical, a C₁₋₆-haloalkyl radical and a hydroxyl radical.

A still more preferred embodiment of the invention is represented by compounds of formula (Ia1′) and (Ia2′):

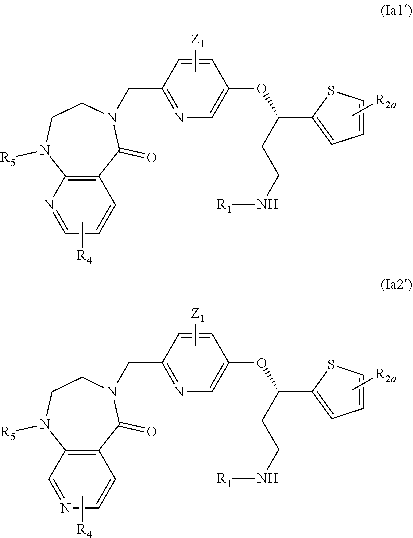

wherein

R₁ is a branched unbranched C₁₋₆ allyl radical, more preferably methyl;

R₂ₐ is a hydrogen atom or a halogen atom, more preferably hydrogen;

Z₁ represents a halogen atom, more preferable fluorine;

R₄ is a hydrogen atom; a halogen atom, more preferably fluorine; a branched or unbranched C₁₋₆-alkoxy radical; or a —NR₄ₐR_{4b} radical where R₄ₐ and R_{4b} are independently a hydrogen atom or a branched or unbranched C₁₋₆-alkyl radical;

R₅ is a branched or unbranched C₁₋₆ alkyl radical, more preferable methyl;

or a pharmaceutically acceptable salt, isomer, prodrug or solvate thereof.

The compounds of the present invention represented by the above-described formula (Ia) may include enantiomers depending on the presence of chiral centers or isomers depending on the presence of double bonds (e.g. Z, E). The single isomers, enantiomers or diastereoisomers and mixtures thereof fall within the scope of the present invention.

In particularly preferred embodiment of the invention the compounds of general formula (I) showing a dual affinity, towards the α2δ-1 subunit of voltage-gated calcium channels (VGCC) and the noradrenaline transporter (NET) are selected from:
[1] (S)-4-((3-Fluoro-5-(3-(methylamino)-1-phenylpropoxy)pyridin-2-yl)methyl)-1-methyl-1,2,3,4-tetrahydro-5H-pyrido[2,3-e][1,4]diazepin-5-one;
[2] (S)-7-fluoro-4-((3-fluoro-5-(1-(3-fluorophenyl)-3-(methylamino)propoxy)pyridin-2-yl)methyl)-1-methyl-1,2,3,4-tetrahydro-5H-pyrido[2,3-e][1,4]diazepin-5-one;
[3] (S)-8-amino-4-((3-fluoro-5-(1-(3-fluorophenyl)-3-(methylamino)propoxy)pyridin-2-yl)methyl)-1-methyl-1,2,3,4-tetrahydro-5H-pyrido[2,3-e][1,4]diazepin-5-one;
[4] (S)-8-(ethylamino)-4-((3-fluoro-5-(1-(3-fluorophenyl)-3-(methylamino)propoxy)pyridine-2-yl)methyl)-1-methyl-1,2,3,4-tetrahydro-5H-pyrido[2,3-e][1,4]diazepin-5-one;
[5] (S)-4-((3-chloro-5-(1-(3-fluorophenyl)-3-(methylamino)propoxy)pyridin-2-yl)methyl)-1-methyl-1,2,3,4-tetrahydro-5H-pyrido[2,3-e][1,4]diazepin-5-one;
[6] (S)-4-((3-fluoro-5-(1-(3-fluorophenyl)-3-(methylamino)propoxy)pyridin-2-yl)methyl)-7-methoxy-1-methyl-1,2,3,4-tetrahydro-5H -pyrido[3,4-e][1,4]diazepin-5-one and
[7] (S)-4-((3-fluoro-5-(1-(3-fluorophen)-3-(methylamino)propoxy)pyridin-2-yl)methyl)-1-methyl-1,2,3,4-tetrahydro-5H-pyrido[2,3-e][1,4]diazepin-5-one;
[8] (S)-4-((3-Fluoro-5-(3-(methylamino)-1-(thiophen-2-yl)propoxy)pyridin-2-yl)methyl)-1-methyl-1,2,3,4-tetrahydro-5H-pyrido[2,3-e][1,4]diazepin-5-one;

or a pharmaceutically acceptable salt, prodrug or solvate thereof.

In a particularly preferred embodiment of the invention the compound of general formula (I) showing a dual affinity, towards the α2δ-1 subunit of voltage-gated calcium channels (VGCC) and the noradrenaline transporter (NET) is:
[8] (S)-4-((3-Fluoro-5-(3-(methylamino)-1-(thiophen-2-yl)propoxy)pyridin-2-yl)methyl)-1-methyl-1,2,3,4-tetrahydro-5H-pyrido[2,3-e][1,4]diazepin-5-one;

or a pharmaceutically acceptable salt, prodrug or solvate thereof.

In another aspect, the invention refers to the processes for obtaining the compounds of general formula (I). Several procedures have been developed for obtaining all the compounds of the invention. Some of them will be explained below in methods A, B and C.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

### Method A

Method A represents a first process for synthesizing compounds according to general formula (I). Method A allows the preparation of compounds of general formula (Ia), that is compounds of general formula (I) where m is 0. There are described two methods for obtaining compounds of general formula (Ia), namely method A1 and A2.

### Method A1

A process is described for the preparation of a compound of general formula (Ia): comprising:

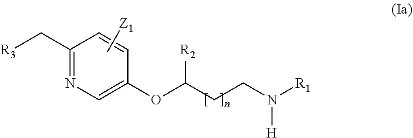

comprising

the reaction of a compound of formula (IIa):

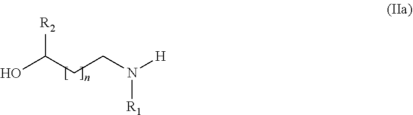

with a compound of formula (IIIa) or (IIIb):

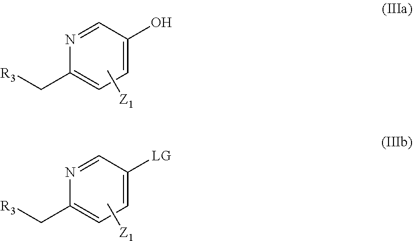

wherein R₁, R₂, R₃, Z₁ and n are as defined before and LG is a suitable leaving group such as chloro, bromo, iodo, mesylate, tosyate, nosylate or triflate.

In the case where the reaction is carried out between a compound of general formula (IIa) with an hydroxyl compound of general formula (IIIa), it is carried out under conventional Mitsunobu conditions by treating an alcohol of general formula (IIa) with a compound of general formula (IIIa) in the presence of an azo compound such as 1,1′-(azodicarbonyl)dipiperidine (ADDP), diisopropylazodicarboxylate (DIAD) or diethyl azodicarboxylate (DEAD) and phosphine such as tributylphosphine or triphenylphoshine. The Mitsunobu reaction is carried out in a suitable solvent, such as toluene or tetrahydrofuran (THF), at a suitable temperature comprised between 0° C. and the reflux temperature, preferably at room temperature, or alternatively, the reactions can be carried out in a microwave reactor.

Whenever the reaction is carried out between a compound of general formula (IIa) and a compound of general formula (IIIb) it is preferably carried out under conventional aromatic nucleophilic substitution conditions by treating an alcohol of general formula (IIa) with a compound of general formula (IIIb) wherein LG represents a leaving group (preferably fluoro), in the presence of a strong base such as sodium hydride or potassium tert-butoxide. The reaction is carried out in a suitable solvent, such as a polar aprotic solvent, preferably dimethylformamide (DMF), dimethylacetamide (DMAC) or dimethylsulfoxide (DMSO); at a suitable temperature comprised between −10° C. and the reflux temperature, preferably at room temperature, or alternatively the reactions can be carried out in a microwave reactor. Alternatively, when LG is triflate, promo or iodo, the compound of general formula (IIIb) can be introduced under cross-coupling conditions, using a Pd or Cu catalyst and a suitable ligand.

Compound of general formula (IIa) is commercially available or can be obtained by reduction of the corresponding ketones, preferably using a hydride source. In addition, the reduction can be performed under asymmetric conditions described in the literature to render chiral compounds of general formula (IIa) in enantiopure form. As a way of example, the chiral reduction can be performed using a hydride source such as borane-tetrahydrofuran complex or borane-dimethyl sulfide complex, in the presence of a Corey-Bakshi-Shibata oxazaborolidine catalyst, in a suitable solvent such as tetrahydrofuran or toluene, at a suitable temperature, preferably comprised between 0° C. and room temperature. Alternatively, using enantiopure B-chlorodiisopinocampheylborane, in a suitable solvent such as tetrahydrofuran, at a suitable temperature, preferably comprised between −40° C. and room temperature.

Alternatively compound of general formula (IIa) can be obtained by deprotection of a compound of general formula (IIa)-P (see scheme 1) protected with any suitable protecting group (P), such as for example Boc (tert-butoxycarbonyl) or Teoc (2-(trimethylsilyl)ethoxycarbonyl). Boc or Teoc deprotection can be effected by any suitable method, such as treatment with an acid, preferably HCl or trifluoroacetic acid in an appropriate solvent such as 1,4-dioxane, dichloromethane (DCM), ethyl acetate or a mixture of an organic solvent and water; alternatively by treatment with ZnBr₂ in an organic solvent, preferably DCM. Alternatively, for Teoc deprotection, by reaction with CsF in an organic solvent, preferably DMF at a temperature range of 20-130° C. alternatively under microwaves irradiation.

Also compound of general formula (IIa) can be obtained by incorporation of the amino group into a compound of general formula (IIa)-LG by an alkylation reaction with compound of general formula (VI) (see scheme 1). The alkylation reaction is carried out in a suitable solvent, such as ethanol, dimethylformamide, dimethylsulfoxide, acetonitrile (ACN) or a mixture of an organic solvent and water, preferably ethanol; optionally in the presence of a base such as K₂CO₃ or triethylamine (TEA); at a suitable temperature comprised between room temperature and the reflux temperature, preferably heating, or alternatively, the reactions can be carried out in a microwave reactor. Additionally, an activating agent such as sodium iodide or potassium iodide can be used.

Compounds of general formula (IIIa), (IIIb) or (VI) are commercially available or can be prepared by conventional methods described in the bibliography.

### Method A2

A further alternative process for the preparation of a compound of general formula (Ia)

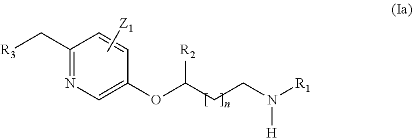

comprises the reaction of a compound of general formula (IV-LG):

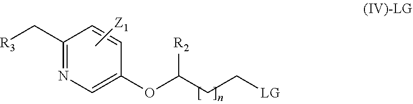

with a compound of general formula (VI):

H₂NR₁ (VI)

wherein R₁, R₂, R₃, Z₁ and n are as defined before and LG represents a suitable leaving group such as chloro, bromo, iodo, mesylate, tosylate, nosylate or triflate.

The alkylation reaction is carried out in a suitable solvent, such as ethanol, dimethylformamide, dimethylsulfoxide, acetonitrile or a mixture of an organic solvent and water, preferably a mixture of ethanol and water; optionally in the presence of a base such as K₂CO₃ or triethylamine; at a suitable temperature comprised between room temperature and the reflux temperature, preferably heating, or alternatively, the reactions can be carried out in a microwave reactor. Additionally, an activating agent such as sodium iodide or potassium iodide can be used.

Compound of general formula (IV)-LG can be prepared by reaction of a compound of general formula (IIb)-LG where LG represents a leaving group (such as chloro, bromo, iodo, mesylate, tosylate, nosylate or triflate) with a compound of general formula (IIIa) (see scheme 1). The reaction is carried out preferably in the presence of a base, such as sodium hydride. The alkylation reaction is carried out in a suitable solvent, such as tetrahydrofuran or dimethylformamide, at a suitable temperature comprised between 0° C. and the reflux temperature, preferably at room temperature.

### Method B

Method B represents a process for synthesizing compounds according to general formula (Ib), namely compounds of general formula (I) where m is 1. There are described two methods for obtaining compounds of general formula (Ib), namely method B1 and B2.

### Method B1

A first process is described preparation of a compound of general formula (Ib):

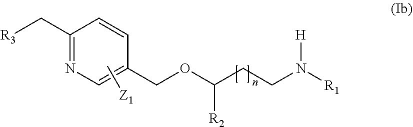

comprising:

a) the reaction between a compound of general formula (IIa):

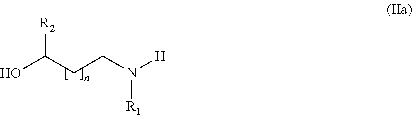

with a compound of general formula (IIIc):

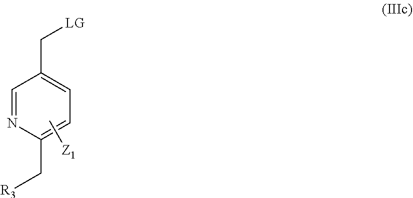

wherein R₁, R₂, R₃, Z₁ and n are as defined before and LG represents a suitable leaving group such as chloro, bromo, iodo, mesylate, tosylate, nosylate or triflate.

The reaction between the compound of general formula (IIa) with an alkylating agent of general formula (IIIc) is carried out in the presence of a strong base such as sodium hydride or potassium tert-butoxide. The alkylation reaction is preferably carried out in a suitable solvent, such as tetrahydrofuran or dimethylformamide, at a suitable temperature comprised between0° C. and the reflux temperature, preferably room temperature, or alternatively, the reactions can be carried out in a microwave reactor. Additionally, an activating agent such as sodium iodide or a phase transfer catalyst such as tetrabutylammonium iodide can be used.

Compound of general formula (IIIc) is commercially available or can be prepared by conventional methods described in the bibliography.

### Method B2

The second method for preparing compounds of general formula (Ib)

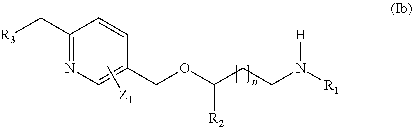

comprises the deprotection of a compound of general formula (V)-P:

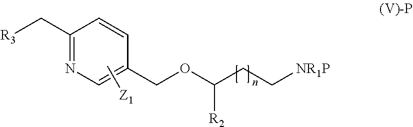

wherein R₁, R₂, R₃, Z₁ and n are as defined before and P represents a protecting group such as, for example, Boc (tert-butoxycarbonyl) or Teoc (2-(trimethylsilyl)ethoxycarbonyl).

Boc or Teoc deprotection can be effected by any suitable method, such as treatment with an acid, preferably HCl or trifluoroacetic acid in an appropriate solvent such as 1,4-dioxane, DCM, ethyl acetate or a mixture of an organic solvent and water; alternatively by treatment with ZnBr₂ in an organic solvent, preferably DCM. Alternatively, for Teoc deprotection, by reaction with CsF in an organic solvent, preferably DMF at a temperature range of 20-130° C., alternatively under microwaves irradiation.

Scheme 1 below summarizes the synthetic routes of methods A (including A1 and A2) and B (including B1 and B2).

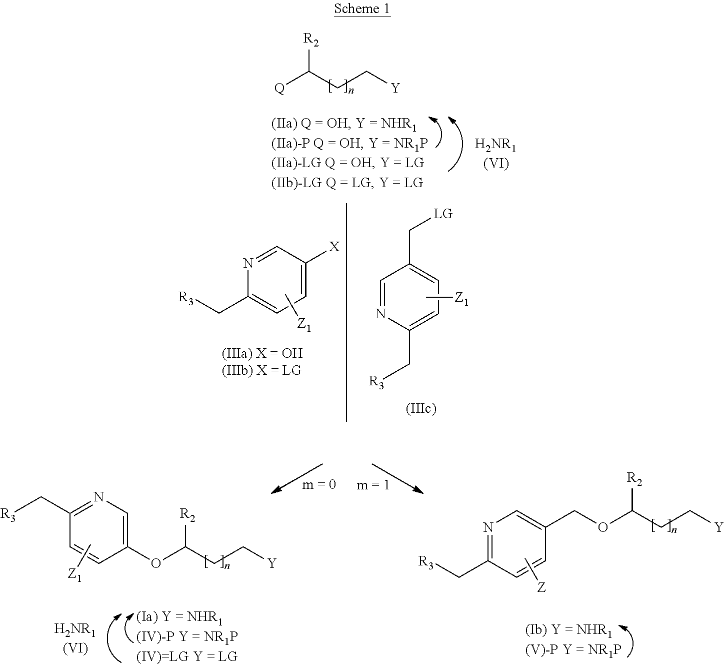

wherein R₁, R₂, R₃, Z₁ and n have the meanings as defined above for a compound of formula (I), LG represents a leaving group (such as chloro, bromo, iodo, mesylate, tosylate, nosylate or triflate) and P represents a protecting group of the amino function.

### Method C

Method C represents the third process for synthesizing compounds according to general formula (I).

In this sense, there is provided a process for the preparation of a compound of general formula (I):

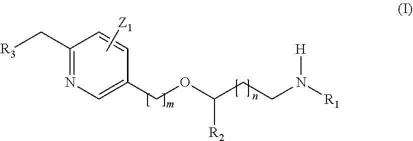

starting from a compound general formula (VII):

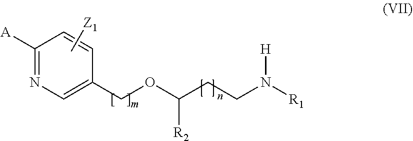

wherein R₁, R₂, R₃, Z₁, m and n are as defined before and where A may represent an aldehyde or a —CH₂-LG group wherein LG represents a suitable leaving group and where the reaction is dependent on the nature of A resulting in that the reaction comprises:
a reductive amination reaction in the presence of a reductive agent, when A is an aldehyde;
a reaction in the presence of a base when A is —CH₂—LG.

As explained above, the reaction of an intermediate of general formula (VII) or its counterparts (VII)-P and (VII)-LG (see scheme 2 below) to give a compound of general formula (I) (or its counterparts (IV/V)-P and (IV/V)-LG, respectively) may be carried out under different reaction conditions, depending on the nature of the groups A:
When A is an aldehyde, by reductive amination reaction in the presence of a reductive reagent, preferably sodium triacetoxyborohydride, in the presence of a base, preferably diisopropylethylamine (DIPEA) or triethylamine (TEA), in an organic solvent, preferably 1,2-dichloroethane (DCE).
When A is a —CH₂-LG group (where LG is a good leaving group as a halogen atom or sulfonate), the reaction may be carried out in the presence of a base, preferably NaH, DIPEA or TEA, in an organic solvent, preferably DMF or THF, at a suitable temperature, preferably in the range of −10 to 100° C. Alternatively, in the presence of tetrabutylammonium iodide (TBAI).

The different synthetic routes including method C as well as reactions for preparing the intermediate compounds for such reactions are depicted in scheme 2:

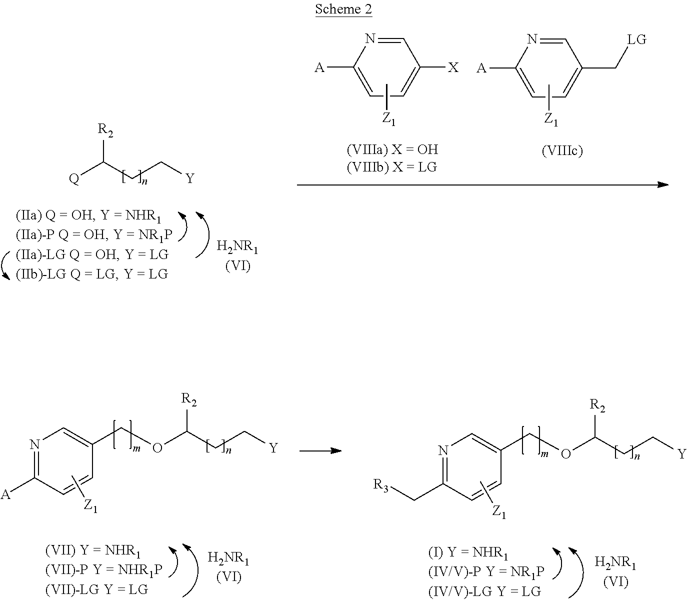

wherein R₁, R₂, R₃, Z₁, m and n have the meaning as defined above, LG represents a leaving group (such as chloro, bromo, iodo, mesylate, tosylate, nosylate or triflate), P represents a protecting group of the amino function and A represents a suitable function to be converted to a group R₃—CH₂—.

Intermediates of type (VII) can be obtained from compounds of general formula (IIa) or (IIb) and reagents of general formula (VIIIa), (VIIIb) or (VIIIc) using the same reaction conditions as described above in methods A and B.

Moreover, compounds of general formula (IV) and (V) can also be obtained by appropriate conversion reactions of functional groups, in one or several steps, using well-known reactions in organic chemistry under standard experimental conditions.

In turn, intermediates of general formula (IIa), (IIa)-P and (IIa)-LG are commercially available or can be obtained by reduction of the corresponding ketones, preferably using a hydride source. In addition, the reduction can be performed under asymmetric conditions described in the literature to render chiral compounds of formula IIa in enantiopure form. As a way of example, the chiral reduction can be performed using a hydride source such as borane-tetrahydrofuran complex or borane-dimethyl sulfide complex, in the presence of a Corey-Bakshi-Shibata oxazaborolidine catalyst, in a suitable solvent such as tetrahydrofuran or toluene, at a suitable temperature, preferably comprised between 0° C. and room temperature. Alternatively, using enantiopure B-chlorodiisopinocampheylborane, in a suitable solvent such as tetrahydrofuran, at a suitable temperature, preferably comprised between −40° C. and room temperature.

The compounds of general formula (IIb-LG) are commercially available or can be obtained from compounds of general formula (IIa-LG) by conventional methods described in the bibliography. For example, using methanesulfonyl chloride in an organic solvent, preferably DCM, in the presence of a base, preferably TEA or DIPEA, at a temperature range of 0° C. and room temperature.

The compounds of general formula (III), (VI) and (VIII) are commercially available or can be prepared by conventional methods described in the bibliography.

Moreover, certain compounds of the present invention can also be obtained starting from other compounds of general formula (I) by appropriate conversion reactions of functional groups, in one or several steps, using well-known reactions in organic chemistry under standard experimental conditions.

In addition, a compound of general formula (I) that shows chirality can also be obtained by resolution of a racemic compound of general formula (I) either by chiral preparative HPLC or by crystallization of a diastereomeric salt or co-crystal. Alternatively, the resolution step can be carried out at a previous stage, using any suitable intermediate.

Turning to another aspect, the invention also relates to the therapeutic use of the compounds of general formula (I). As mentioned above, compounds of general formula (I) show a strong affinity both to the subunit α2δ and more preferably to the α2δ-1 subunit of voltage-gated calcium channels as well as to the noradrenaline transporter (NET) and can behave as agonists, antagonists, inverse agonists, partial antagonists or partial agonists thereof. Therefore, compounds of general formula (I) are useful as medicaments.

They are suitable for the treatment and/or prophylaxis of diseases and/or disorders mediated by the subunit α2δ, especially the α2δ-1 subunit of voltage-gated calcium channels and/or the noradrenaline transporter (NET). In this sense, compounds of formula (I) are suitable for the treatment and/or prophylaxis of pain, especially neuropathic pain, inflammatory pain, and chronic pain or other pain conditions involving allodynia and/or hyperalgesia, depression, anxiety and attention-deficit-/hyperactivity disorder (ADHD).

The compounds of formula (I) are especially suited for the treatment of pain, from medium to severe pain, visceral pain, chronic pain, cancer pain, migraine, inflammatory pain, acute pain or neuropathic pain, allodynia or hyperalgesia. This may include mechanical allodynia or thermal hyperalgesia.

PAIN is defined by the international Association for the Study of Pain (IASP) as “an unpleasant sensory and emotional experience associated with actual or potential tissue damage, or described in terms of such damage (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment compounds of the invention are used for the treatment and/or prophylaxis of neuropathic pain and more specifically for the treatment and/or prophylaxis of hyperpathia.

A related aspect of the invention refers to the use of compounds of formula (I) for the manufacture of a medicament for the treatment and/or prophylaxis of disorders and diseases mediated by the subunit α2δ, especially the α2δ-1 subunit of voltage-gated calcium channels and/or the noradrenaline transporter (NET), as explained before.

Another related aspect of the invention refers to a method for the treatment and/or prophylaxis of disorders and diseases mediated by the subunit α2δ, especially the α2δ-subunit of voltage-gated calcium channels and/or the noradrenaline transporter (NET), as explained before comprising the administration of a therapeutically effective amount of a compound of general formula (I) to a subject in need thereof.

Another aspect of the invention is a pharmaceutical composition, which comprises at least a compound of general formula (I) or a pharmaceutically acceptable salt, prodrug, isomer or solvate thereof, and a east pharmaceutically acceptable carrier, additive, adjuvant or vehicle.

The pharmaceutical composition of the invention can be formulated as a medicament in different pharmaceutical forms comprising at least a compound binding to the subunit α2δ, especially the α2δ-1 subunit of voltage-gated calcium channels and the noradrenaline transporter (NET) and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenteral, for example pulmonary, nasally, rectally and/or intravenously.

Preferably, the composition is suitable for oral or parenteral administration, more preferably for oral, intravenous, intraperitoneal, intramuscular, subcutaneous, intrathekal, rectal, transdermal, transmucosal or nasal administration.

The composition of the invention can be formulated for oral administration in any form preferably selected from the group consisting of tablets, drageés, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tableting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the British and US Pharmacopoeias and reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

In the next preparation examples, the preparation of both intermediates compounds as well as compounds according to the invention is disclosed.

The following abbreviations are used:

### Examples

The preparation of examples according to the invention is disclosed.

The following abbreviations are used:

### Acetonitrile: ACN

### Aq: Aqueous

### CH: Cyclohexane

### DCM: Dichloromethane

### DIPEA: N,N-Diisopropylethylamine

### DMA: N,N-Dimethylacetamide

EtOAc: Ethyl acetate

### h: Hour/s

HPLC: High-performance liquid chromatography
MS: Mass spectrometry

### Min: Minutes

Ret: Retention time
rt: Room temperature

### Sat: Saturated

TBAI: Tetrabutylammonium iodide
TFA: Trifluoroacetic acid

### THF: Tetrahydrofuran

The following methods were used to generate the HPLC-MS data:

Method A: Column Eclipse XDB-C18 4.6×150 mm, 5 μm; flow rate 1 mL/min; A: H₂ O (0.05% TFA); B: ACN; Gradient: 5% to 95% B in 7 min, isocratic 95% B 5 min.

Method B: Column Zorbax SB-C18 2.1×50 mm, 1.8 μm; flow rate 0.5 mL/min; A: H₂O (0.1% formic acid); B: ACN (0.1% formic acid); Gradient; 5% to 95% B in 4 min, isocratic 95% B 4 min.

### Example 1

### (S)-4-((3-Fluoro-5-(3-(methylamino)-1-phenylpropoxy)pyridin-2-yl)methyl)-1-methyl-1,2,3,4-tetrahydro-5H-pyrido[2,3-e][1,4]diazepin-5-one

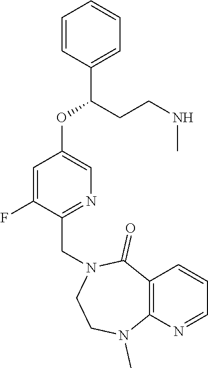 [SMILES: CNCC[C@H](Oc1cnc(CN2CCN(C)c3ncccc3C2=O)c(F)c1)c1ccccc1]

a) Methyl (S)-5-(3-((tert-butoxycarbonyl)(methyl)amino)-1-phenylpropoxy)-3-fluoropicolinate. To a solution of tert-butyl (S)-(3-hydroxy-3-phenylpropyl) (methyl)carbamate (650 mg, 2.45 mmol) and methyl 3,5-difluoropicolinate (848 mg, 4.90 mmol) in DMA (13.6 mL), NaH (60% suspension in mineral oil, 147 mg, 3.67 mmol) was added and the mixture was stirred at rt for 2.5 h. Water was added, extracted with EtOAc, dried with Na₂SO₄, filtered and concentrated under vacuum. Purification by flash chromatography, silica gel, gradient from CH to 100% EtOAc afforded the title product (509 mg, 50% yield) as a mixture of two regioisomers (7:3).

HPLC (Method B): Ret, 5.3 min; ESI⁺-MS m/z, 419.2 (M+H).

b) tert-Butyl (S)-(3-((5-fluoro-6-(hydroxymethyl)pyridin-3-yl)oxy)-3-phenylpropyl)(methyl)carbamate. To a solution of the compound obtained in step a (505 mg, 1.20 mmol) in diethylether (8 mL) at rt under Ar atmosphere, lithium tri-tert-butoxyaluminium hydride (1M solution in THF, 8 mL, 8.0 mmol) was added and the mixture was heated at 50° C. in a sealed tube for 16 h. The reaction mixture was cooled at 0° C., EtOAc and sat solution of Rochelle salt were added and the mixture was vigorously stirred for 1 h. The aq phase was separated and extracted with EtOAc. The combined organic layers were dried over Na₂SO₄, filtered and concentrated under vacuum. Purification by flash chromatography, silica gel, gradient from CH to 100% EtOAc afforded the title product (347 mg, 73% yield) as a mixture of two regioisomers (7:3).

HPLC (Method B): Ret, 5.8 min; ESI⁺-MS m/z, 391.2 (M+H).

c) tert-Butyl (S)-(3-((6-(chloromethyl)-5-fluoropyridin-3-yl)oxy)-3-phenylpropyl)(methyl)carbamate. To a solution of the compound obtained in step b (350 mg, 0.89 mmol) and DIPEA (0.313 mL, 1.79 mmol) in DCM (7.5 mL) cooled at 0° C., methanesulfonyl chloride (0.091 mL, 1.16 mmol) was added dropwise and the reaction mixture was stirred at rt for 16 h. Cold water was added, extracted with DCM, washed with cold NaCl sat solution, dried over Na₂SO₄, filtered and concentrated under vacuum to afford the title product that was used in the next step without further purification (360 mg, 98% yield).

d) tert-Butyl (S)-(3-((5-fluoro-6-((1-methyl-5-oxo-1,2,3,5-tetrahydro-4H-pyrido[2,3-e][1,4]diazepin-4-yl)methyl)pyridin-3-yl)oxy)-3-phenylpropyl)(methyl)carbamate. To a solution of 1-methyl-1,2,3,4-tetrahydro-5H-pyrido[2,3-e][1,4]diazepin-5-one (141 mg, 0.79 mmol) in DMF (6 mL) cooled at 0° C. NaH (60% suspension in mineral oil, 48 mg, 1.2 mmol) was added and the mixture was stirred at rt for 30 min. The reaction mixture was cooled again at 0° C. and a solution of the compound obtained in step c (358 mg, 0.87 mmol) in DMF (4 mL) and TBAI (29 mg, 0.08 mmol) were added and the reaction mixture was stirred at rt for 4 h. Water was added, extracted with EtOAc and the organic layer was dried with Na₂SO₄, filtered and concentrated under vacuum. Purification by flash chromatography, silica gel, gradient from CH to 40% acetone afforded the title product (227 mg, 55% yield) as single regioisomers.

HPLC (Method B): Ret, 6.9 min; ESI⁺-MS m/z, 550.3 (M+H).

e) Title compound. To a solution of the compound prepared in step d (269 mg, 0.49 mmol) in dioxane (1 mL) at 0° C., HCl 4M solution in dioxane (1.71 mL, 6.8 mmol) was added and the mixture was stirred at 0° C. for 2 h. The reaction mixture was concentrated to dryness under vacuum to afford the title product (202 mg, 92% yield).

HPLC (Method A); Ret, 4.43 min; ESI⁺-MS m/z, 450.2 (M+H).

This method was used for the preparation of Ex 2-7 using suitable starting materials:

| | | Chemical | | Ret | |
|---|---|---|---|---|---|
| EX | Structure | name | Method | (min) | MS |
| 2 | 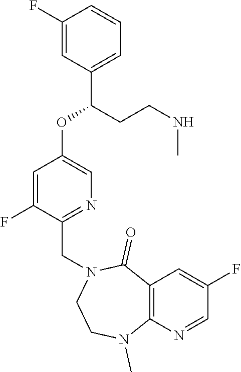 [SMILES: CNCC[C@H](Oc1cnc(CN2CCN(C)c3ncc(F)cc3C2=O)c(F)c1)c1cccc(F)c1] | (S)-7-fluoro-4-((3- fluoro-5-(1-(3- fluorophenyl)-3- (methylamino) propoxy)pyridin-2- yl)methyl)-1- methyl-1,2,3,4- tetrahydro-5H- pyrido[2,3- e][1,4]diazepin-5- one | A | 5.58 | 486.2 (M + H) |
| 3 | 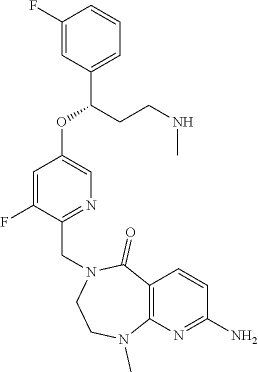 [SMILES: CNCC[C@H](Oc1cnc(CN2CCN(C)c3nc(N)ccc3C2=O)c(F)c1)c1cccc(F)c1] | (S)-8-amino-4-((3- fluoro-5-(1-(3- fluorophenyl)-3- (methylamino) propoxy)pyridin-2- yl)methyl)-1- methyl-1,2,3,4- tetrahydro-5H- pyrido[2,3- e][1,4]diazepin-5- one | A | 4.50 | 483.3 (M + H) |
| 4 | 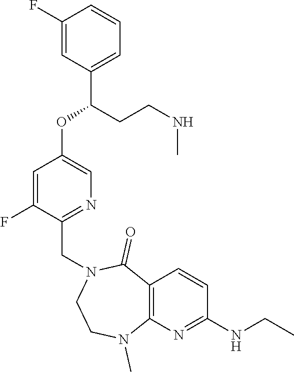 [SMILES: CCNc1ccc2c(n1)N(C)CCN(Cc1ncc(O[C@@H](CCNC)c3cccc(F)c3)cc1F)C2=O] | (S)-8-(ethylamino)- 4-((3-fluoro-5-(1-(3- fluorophenyl)-3- (methylamino) propoxy)pyridin-2- yl)methyl)-1- methyl-1,2,3,4- tetrahydro-5H- pyrido[2,3- e][1,4]diazepin-5- one | A | 4.80 | 511.3 (M + H) |
| 5 | 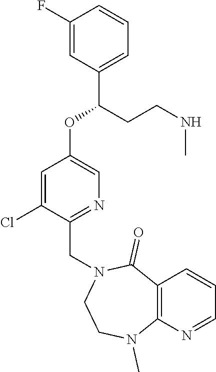 [SMILES: CNCC[C@H](Oc1cnc(CN2CCN(C)c3ncccc3C2=O)c(Cl)c1)c1cccc(F)c1] | (S)-4-((3-chloro-5- (1-(3-fluorophenyl)- 3- (methylamino) propoxy)pyridin-2- yl)methyl)-1- methyl-1,2,3,4- tetrahydro-5H- pyrido[2,3- e][1,4]diazapin-5- one | A | 4.67 | 484.2 (M + H) |
| 6 | 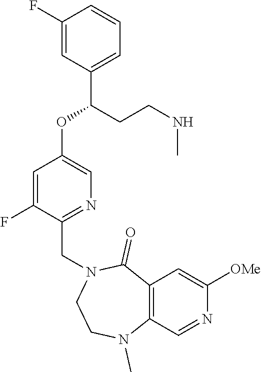 [SMILES: CNCC[C@H](Oc1cnc(CN2CCN(C)c3cnc(OC)cc3C2=O)c(F)c1)c1cccc(F)c1] | (S)-4-((3-fluoro-5- (1-(3-fluorophenyl)- 3- (methylamino) propoxy)pyridin-2- yl)methyl)-7- methoxy-1-methyl- 1,2,3,4-tetrahydro- 5H-pyrido[3,4- e][1,4]diazepin-5- one | A | 5.47 | 498.3 (M + H) |
| 7 | 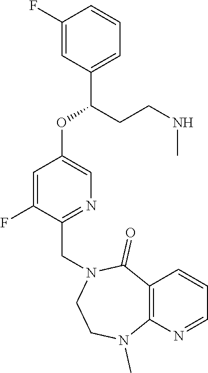 [SMILES: CNCC[C@H](Oc1cnc(CN2CCN(C)c3ncccc3C2=O)c(F)c1)c1cccc(F)c1] | (S)-4-((3-fluoro-5- (1-(3-fluorophenyl)- 3- (methylamino) propoxy)pyridin-2- yl)methyl)-1- methyl-1,2,3,4- tetrahydro-5H- pyrido[2,3- e][1,4]diazepin-5- one | A | 4.52 | 468.2 (M + H) |

### Example 8

### (S)-4-((3-Fluoro-5-(3-(methylamino)-1-(thiophen-2-yl)propoxy)pyridin-2-yl)methyl)-1-methyl-1,2,3,4-tetrahydro-5H-pyrido[2,3-e][1,4]diazepin-5-one

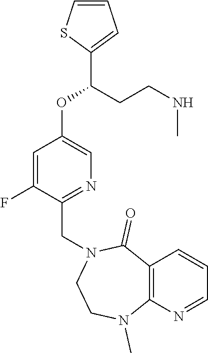 [SMILES: CNCC[C@H](Oc1cnc(CN2CCN(C)c3ncccc3C2=O)c(F)c1)c1cccs1]

a) 4-((3,5-Difluoropyridin-2-yl)methyl)-1-methyl-1,2,3,4-tetrahydro-5H-pyrido[2,3-e][1,4]diazepin-5-one. To a solution of 1-methyl-1,2,3,4-tetrahydro-5H-pyrido[2,3-e] [1,4]diazepin-5-one (260 mg, 1.46 mmol) in DMF (6 mL) at 0° C., NaH (60% suspension in mineral oil, 88 mg, 2.20 mmol) was added and the mixture was stirred at rt for 30 min. The reaction mixture was cooled at 0° C., a solution of 2-(chloromethyl)-3,5-difluoropyridine (288 mg, 1.76 mmol) in DMF (5.5 mL) and TBAI (54 mg, 0.147 mmol) were added and the mixture was warmed at rt and stirred for 20 h. Water was added and extracted with EtOAc. The organic layer was dried over Na₂SO₄, filtered and concentrated. Purification by flash chromatography, gradient from CH to 100% EtOAc afforded the title product (395 mg, 88% yield).

HPLC (Method B): Ret, 0.43 min; ESI⁺-MS m/z, 304.5 (M+H).

b) Title compound. To a solution of (S)-3-(methylamino)-1-(thiophen-2-yl)propan-1-ol (270 mg, 1.577 mmol) in DMF (11.5 mL) at 0° C., KOtBu (265 mg, 2.36 mmol) was added and the mixture was stirred at rt for 15 min. The reaction mixture was cooled at 0° C., a solution of the compound prepared in step a (408 mg, 1.34 mmol) in DMF (2 mL) was added and the mixture was stirred at rt for 20 h. Water was added and extracted with EtOAc. The organic layer was dried over Na₂SO₄, filtered and concentrated to afforded a mixture of the title product and the regioisomer that was purified by semipreparative HPLC: Chiralpak IC 250×4.6 mm, 5 μm, MeOH:DEA (100:1), 1 ml/min, ret 12.25 min.

HPLC (Method A): Ret, 4.28 min; ESI⁺-MS m/z, 456.2 (M+H).

### Pharmacological Data

### Binding Assay to Human α2δ-1 Subunit of Cav2.2 Calcium Channel

Human α2δ-1 enriched membranes (2.5 μg) were incubated with 15 nM of radiolabeled [3H]-Gabapentin in assay buffer containing Hepes-KOH 10 mM, pH 7.4. NSB (non-specific binding) was measured by adding 10 μM pregabalin. The binding of the test compound was measured at either one concentration (% inhibition at 1 or 10 μM) or five different concentrations to determine affinity values (Ki). After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μl of scintillation cocktail were added to each well before radioactivity reading. Readings were performed in a Trilux 1460 Microbeta radioactive counter (Perkin Elmer).

### Binding Assay to Human Norepinephrine Transporter (NET)

Human norepinephrine transporter (NET) enriched membranes (5 μg) were incubated with 5 nM of radiolabeled [3H]-Nisoxetin in assay buffer containing 50 mM Tris-HCl, 120 mM NaCl, 5 mM KCl, pH 7.4. NSB (non specific binding) was measured by adding 10 μM of desipramine. The binding of the test compound was measured at either one concentration (% inhibition at 1 or 10 μM) or five different concentrations to determine affinity values (Ki). After 60 min incubation at 4° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, 0.9% NaCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

The following scale has been adopted for representing the binding to the α2δ-1 receptor expressed as Ki:
+ Ki-α2δ-1>=3000 nM
++ 500 nM<Ki-α2δ-1<3000 nM
+++ 100 nM<Ki-α2δ-1<500 nM
++++ Ki-α2δ-1<100 nM

Preferably, when Kᵢ(α₂δ-1)>3000 nM, the following scale has been adopted for representing the binding to the α₂δ-1 subunit of voltage-gated calcium channels:
+ Kᵢ(α₂δ-1)>3000 nM or inhibition ranges between 1% and 50%.

Regarding the NET receptor, the following scale has been adopted for representing the binding expressed as Ki:
+ Ki-NET>=1000 nM
++ 500 nM<Ki-NET<1000 nM
+++ 100 nM<Ki-NET<500 nM
++++ Ki-NET<100 nM

Preferably, when Kᵢ (NET)>1000 nM, the following scale has been adopted for representing the binding to the NET-receptor:
+ Kᵢ (NET)>1000 nM or inhibitor ranges between 1% and 50 %.

The Ki results for the α2δ-1 subunit of the voltage-gated calcium channel and the NET transporter are shown In Table 1:

TABLE 1

| | Ki(nM) | Ki(nM) |
|---|---|---|
| Example | NET | alpha2delta |
| number | Hum | Hum |
| 1 | ++++ | ++++ |
| 2 | ++++ | +++ |
| 3 | ++++ | ++++ |
| 4 | ++++ | ++++ |
| 5 | ++++ | ++++ |
| 6 | ++++ | ++++ |
| 7 | ++++ | +++ |
| 8 | ++++ | +++ |

1-17. (canceled)

18. A compound of general formula (I): 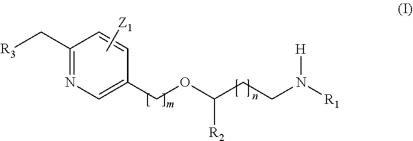
wherein:
R₁ is a branched or unbranched C₁₋₆ alkyl radical or a C₁₋₆ haloalkyl radical;
R₂ is a 6-membered aryl optionally substituted by a halogen atom, a branched or unbranched C₁₋₆-alkyl radical, a branched or unbranched C₁₋₆-alkoxy radical, a C₁₋₆₋haloalkoxy radical, a C₁₋₆-haloalkyl radical or a hydroxyl radical; or 5 or 6-membered heteroaryl having at least one heteroatom selected from N, O and S;
n and m are independently 0 or 1;
Z₁ is a hydrogen atom; a branched or unbranched C₁₋₆-alkyl radical; a halogen atom; a branched or unbranched C₁₋₆-alkoxy radical; a C₁₋₆-haloalkyl radical; or a C₁₋₆₋haloalkoxy radical;
R₃ represents one of the following moieties: 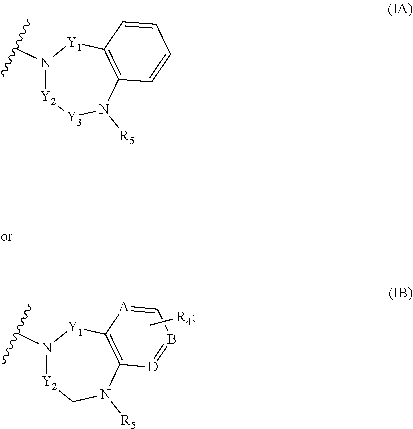
wherein
Y₁, Y₂ and Y₃ are independently —CH₂— or —CH—;
one or two of A, B and D represent —N— and the others represent —C— or —CH—;
R₄ is a hydrogen atom, a branched or unbranched C₁₋₆-alkyl radical; a halogen atom; a branched or unbranched C₁₋₆-alkoxy radical; a C₁₋₆-haloalkyl radical; or a —NR₄ₐR_{4b} radical wherein R₄ₐ and R_{4b} independently represent a hydrogen atom or a branched or unbranched C₁₋₆-alkyl radical;
R₅ is a hydrogen atom; a branched or unbranched C₁₋₆ alkyl radical; or a —C(O)—CH₂—NR₆ₐR_{6b} radical wherein R₅ₐ and R_{5b} independently represent a hydrogen atom or a branched or unbranched C₁₋₆-alkyl radical;
or a pharmaceutically acceptable salt, isomer, prodrug or solvate thereof.

19. The compound according to claim 18, wherein R₁ is a branched or unbranched C₁₋₆ alkyl radical.

20. The compound according to claim 19, wherein R₁ is methyl.

21. The compound according to claim 18, wherein R₂ is a phenyl radical optionally substituted by a halogen atom, a branched or unbranched C₁₋₆-alkyl radical, a branched or unbranched C₁₋₆-alkoxy radical, a C₁₋₆₋haloalkoxy radical, a C₁₋₆-haloalkyl radical or a hydroxyl radical; or wherein R₂ is a thienyl radical optionally substituted by a halogen atom, a branched or unbranched C₁₋₆-alkyl radical, a branched or unbranched C₁₋₆-alkoxy radical, a C₁₋₆₋haloalkoxy radical, a C₁₋₆-haloalkyl radical or a hydroxyl radical.

22. The compound according to claim 21, wherein R₂ is a phenyl radical which is unsubstituted or substituted by a halogen atom or R₂ is an unsubstituted thienyl radical.

23. The compound according to claim 22, wherein R₂ is a phenyl radical which is substituted by a fluorine atom.

24. The compound according to claim 18, wherein R₃ is selected from:

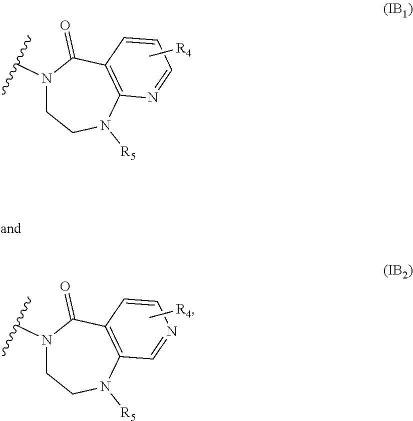

25. The compound according to claim 18, wherein Z₁ represents a halogen atom.

26. The compound according to claim 25, wherein Z₁ represents fluorine or chlorine.

27. The compound according to claim 18, wherein R₄ is a hydrogen atom; a halogen atom; a branched or unbranched C₁₋₆-alkoxy radical; or a —NR₄ₐR_{4b} radical, wherein R₄ₐ and R_{4b} are independently a hydrogen atom or a branched or unbranched C₁₋₆-alkyl radical.

28. The compound according to claim 27, wherein R₄ is fluorine; methoxy; or a —NR₄ₐR_{4b} radical, wherein R₄ₐ and R_{4b} are independently a hydrogen atom or methyl.

29. The compound according to claim 18, wherein R₅ is a branched or unbranched C₁₋₆ alkyl radical.

30. The compound according to claim 29, wherein R₅ is methyl.

31. The compound according to claim 18, which is a compound of formula (Ia) 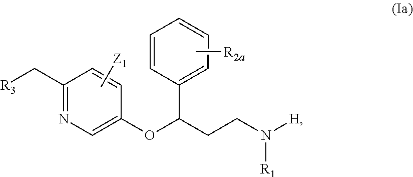
wherein R₁, R₃ and Z₁ are as defined in claim 18, and R₂ₐ is selected from a hydrogen, a halogen atom, a branched or unbranched C₁₋₆-alkyl radical, a branched or unbranched C₁₋₆-alkoxy radical, a C₁₋₆ haloalkoxy radical, a C₁₋₆-haloalkyl radical and a hydroxyl radical;
or a compound of formula (Ia′) 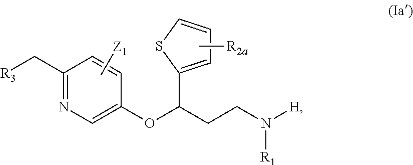
wherein R₁, R₃ and Z₁ are as defined in claim 18, and R₂ₐ is selected from a hydrogen, a halogen atom, a branched or unbranched C₁₋₆-alkyl radical, a branched or unbranched C₁₋₆-alkoxy radical, a C₁₋₆₋haloalkoxy radical, a C₁₋₆-haloalkyl radical and a hydroxyl radical.

32. The compound according to claim 18, which is a compound of formula (Ia1), formula (Ia2), formula (Ia1′) or formula (Ia2′): 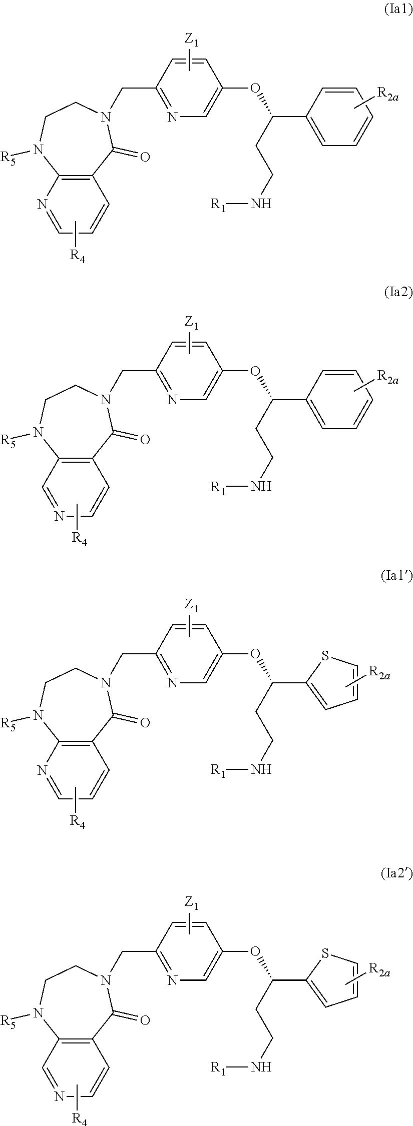
wherein R₁, R₄, R₅ and Z₁ are as defined in claim 18, and R₂ₐ is selected from a hydrogen, a halogen atom, a branched or unbranched C₁₋₆-alkyl radical, a branched or unbranched C₁₋₆-alkoxy radical, a C₁₋₆₋haloalkoxy radical, a C₁₋₆-haloalkyl radical and a hydroxyl radical.

33. The compound according to claim 18, which is selected from:
(S)-4-((3-Fluoro-5-(3-(methylamino)-1-phenylpropoxy)pyridin-2-yl)methyl)-1-methyl-1,2,3,4-tetrahydro-5H-pyrido[2,3-e][1,4]diazepin-5-one;
(S)-7-fluoro-4-((3-fluoro-5-(1-(3-fluorophenyl)-3-(methylamino)propoxy)pyridin-2-yl)methyl)-1-methyl-1,2,3,4-tetrahydro-5H-pyrido[2,3-e][1,4]diazepin-5-one;
(S)-8-amino-4-((3-fluoro-5-(1-(3-fluorophenyl)-3-(methylamino)propoxy)pyridin-2-yl)methyl)-1-methyl-1,2,3,4-tetrahydro-5H-pyrido[2,3-e][1,4]diazepin-5-one;
(S)-8-(ethylamino)-4-((3-fluoro-5-(1-(3-fluorophenyl)-3-(methylamino)propoxy)pyridine-2-yl)methyl)-1-methyl-1,2,3,4-tetrahydro-5H-pyrido[2,3-e][1,4]diazepin-5-one;
(S)-4-((3-chloro-5-(1-(3-fluorophenyl)-3-(methylamino)propoxy)pyrridin-2-yl)methyl)-1-methyl-1,2,3,4-tetrahydro-5H-pyrido[2,3-e][1,4]diazepin-5-one;
(S)-((3-fluoro-5-(1-(3-fluorophenyl)-3-(methylamino)propoxy)pyridin-2-yl)methyl)-7-methoxy-1-methyl-1,2,3,4-tetrahydro-5H-pyrido[3,4-e][1,4]diazepin-5-one;
(S)-4-((3-fluoro-5-(1-(3-fluorophenyl)-3-(methylamino)propoxy)pyridin-2-yl)methyl)-1-methyl-1,2,3,4-tetrahydro-5H-pyrido[2,3-e][1,4]diazepin-5-one;
(S)-4-((3-Fluoro-5-(3-(methylamino)-1-(thiophen-2-yl)propoxy)pyridin-2-yl)methyl)-1-methyl-1,2,3,4-tetrahydro-5H-pyrido[2,3-e][1,4]diazepin-5-one;
and pharmaceutically acceptable salts, prodrugs and solvates thereof.

34. A process for the preparation of a compound of general formula (Ia): 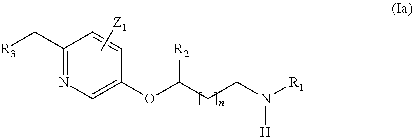
comprising:
a) reaction of a compound of formula (IIa) 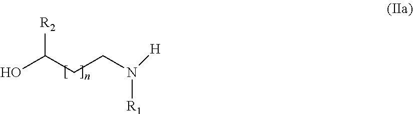
with a compound of formula (IIIa) or (IIIb): 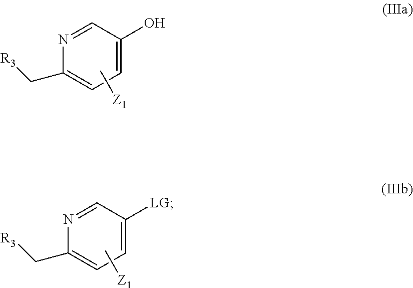
or
b) reaction of a compound of formula (IV)-LG: 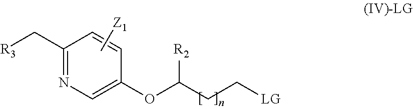
with a compound of formula (VI):
H₂NR1 (VI)
wherein R₁, R₂, R₃, Z₁ and n are as defined in claim 18, and LG represents a leaving group.

35. A process for the preparation of a compound of general formula (Ib): 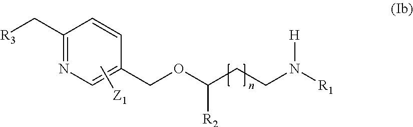
comprising
a) reaction between a compound of formula (IIa): 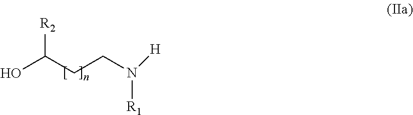
and a compound of formula (IIIc): 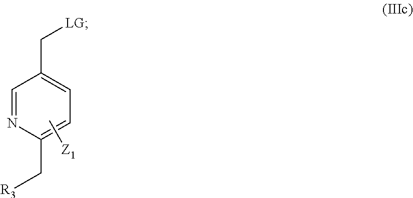
or
b) deprotection of a compound of formula (V)-P: 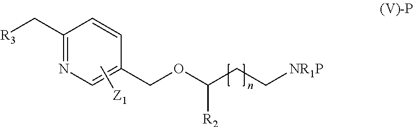
wherein R₁, R₂, R₃, Z₁ and n are as defined in claim 18, LG represents a leaving group, and P represents a protecting group.

36. A process for the preparation of a compound of general formula (I): 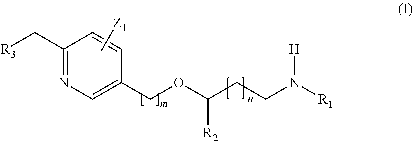
starting from a compound of formula (VII): 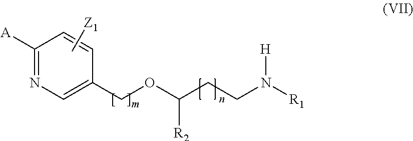
wherein R₁, R₂, R₃, Z₁, m and n are as defined in claim 18, and wherein A represents an aldehyde or a —CH₂-LG group, wherein LG represents a suitable leaving group, and wherein the reaction is dependent on the nature of A, resulting in that the reaction comprises:
a reductive amination reaction in the presence of a reductive agent, when A is an aldehyde; or
a reaction in the presence of a base, when A is —CH₂-LG.

37. A method for the treatment and/or prophylaxis of diseases and/or disorders mediated by the subunit α2δ, including the α2δ-1 subunit, of voltage-gated calcium channels and/or the noradrenaline transporter (NET) in a subject in need thereof, comprising administration of an effective amount of the compound according to claim 18.

38. The method according to claim 37, wherein the disease or disorder is selected from medium to severe pain, visceral pain, chronic pain, cancer pain, migraine, inflammatory pain, acute pain or neuropathic pain or other pain conditions involving allodynia and/or hyperalgesia, depression, anxiety and attention-deficit-/hyperactivity disorder.

39. A pharmaceutical composition comprising the compound according to claim 18, or a pharmaceutically acceptable salt, isomer, prodrug or solvate thereof, and at least a pharmaceutically acceptable carrier, additive, adjuvant or vehicle.
